(19) United States
(12) Patent Application Publication
(10) Pub. No.: US 2023/0021229 A1
(43) Pub. Date: Jan. 19, 2023

(54) METHOD AND DATA PROCESSING DEVICE FOR PROCESSING GENETIC DATA

(71) Applicant: Fraunhofer-Gesellschaft zur Foerderung der angewandten Forschung e. V., Muenchen (DE)

(72) Inventors: Heiko ZIMMERMANN, Sulzbach (DE); Sabine MUELLER, Sulzbach (DE)

(21) Appl. No.: 17/784,720

(22) PCT Filed: Dec. 16, 2020
(86) PCT No.: PCT/EP2020/086414

§ 371 Date: Jun. 13, 2022

(30) Foreign Application Priority Data
Dec. 20, 2019 (DE) 102019135380.7

(51) Int. Cl. G16B 50/40; G16B 50/30; G06N 3/12; H04L 9/32

(52) U.S. Cl. CPC G16B 50/40; G16B 50/30; G06N 3/123; H04L 9/3239

(57) ABSTRACT

A method for processing genetic data, which comprise a series of sequence elements each representing a biomolecule, comprises the steps of forming sequence fragments (S2), wherein each sequence fragment comprises a section of the series of sequence elements having a fragment length of at least two sequence elements, applying a coding function to each of the sequence fragments in order to generate a multiplicity of encrypted fragment data items (S3) winch are each assigned to one of the sequence fragments, and storing the encrypted fragment data (S4), wherein the sequence fragments are formed in such a manner that the sections of the series of sequence elements overlap and each sequence element is included in at least two sequence fragments. A description is also given of a data processing device for processing genetic data and a method for querying a database containing encrypted fragment data which were generated and stored using the method for processing genetic data.

The invention relates to a method and a data processing apparatus for processing, in particular for encrypting, genetic data which represents a series of biomolecules, for example data from nucleotide, amino acid and/or protein sequences. The invention also relates to methods for querying a database containing encrypted genetic data which was generated and stored with said method. Applications of the invention are in the fields of bioinformatics, medicine, cell biology, stem cell technology, pharmacology and/or biotechnology, particularly in the processing of genetic data.

It is generally known that over the last few years by means of effective sequencing techniques, the possibilities for recording and storing genetic data and the extent of the genetic data stored in databases of clinical facilities has significantly increased. For example, genetic data is obtained in a clinical facility from a plurality of examined persons and is stored in conjunction with other data regarding the persons, for example, identification data and data regarding living conditions and/or the state of health of the persons.

These data are of interest not only for diagnostic and therapeutic purposes in the examination and/or treatment of the persons in question. Rather, the data represent a valuable reservoir of information for research and development, for example, in pharmacology. Genetic data can provide information regarding the causes of diseases or disease mechanisms. Genetic data also enable personalized treatments or the development of behavioral or nutritional recommendations and their application adapted to patients individually. There also exists an interest, for research, in access to the genetic data, for example to identify specific individuals with a predetermined genetic disposition (and, where relevant, particular disease and lifestyle conditions) or cell samples from these individuals for targeted investigations of, for example, pharmacological formulations, for example, as a disease model, or for analyses of causes of diseases.

There therefore exists an interest in searching through stored genetic data of a large number of individuals for the occurrence of predetermined features, for example predetermined amino acid sequences and in retrieving the genetic data of the individuals thereby identified and also in making use of it for further investigations.

In the searching and processing of clinically, or otherwise, obtained individual genetic data and also in the shared use of the data (data sharing), in particular in international cooperations, however, the following problems arise.

The human genome possesses approximately 3 billion base pairs. In the investigation of the data of a large number of individuals, for example, tens of thousands of patients, there arise extremely large quantities of data the searching of which for particular search sequences or combinations of search sequences is extremely laborious. There therefore exists an interest in improving the effectiveness (e.g. energy usage and/or duration) of searching through genetic data.

A further restriction in the searching of genetic data lies in the interest of individuals in protecting their data. Since genetic data defines the inherited and/or acquired genetic properties of a person, it represents unique and sensitive information. Nowadays it is assumed that even after a separation of the genetic data from identification data of the associated person, it is still possible to match the data to a particular person. A thorough anonymization of genetic data would require its falsification, whereafter however no further reliable investigation of the data would be possible. Genetic data can therefore at most be pseudonymized, but not thoroughly anonymized.

Therefore for the operation of a database with genetic data, data security (protection against loss, misuse, manipulation and/or other threats) is a substantial requirement. Person-related data are subject to a legally regulated protection against misuse which is formulated, for example, in Germany with the General Data Protection Regulation (DSGVO).

Due to the legal rules on data protection, typically access by third parties to databases with clinically obtained genetic data is excluded, in particular interrupted physically. Due to the inherently precluded or complicated anonymization of genetic data, neither open access via a data network, nor a conditional access for authorized enquiries is possible. In order to be able still to use the potential of person-related genetic data in research and development or for other investigative purposes while ensuring data protection, an interest exists in a new approach to the handling of genetic data.

It is known to store genetic data encrypted for compression purposes. The encryption can take place, for example, by using hash functions. It is proposed by A. Mehta et al. in “DNA compression using hash based data structure” in the “International Journal of Information and Knowledge Management”, 2010, vol. 2, No. 2, pp. 383-386, to save storage space by means of a binary encoding of a DNA sequence. The DNA sequence is fragmented into successive non-overlapping portions and encoded in bits by means of a hash function. There results a shorter sequence of bits which are stored together with a hash table as an alphabet (a “look-up” table). In the hash table, each DNA fragment is mapped to a character. Thus, in the method by A. Mehta et al., a compression of the genetic data is indeed achieved. With separate storage of the hash table, advantages for data protection could even be achieved. What is disadvantageous, however, is that the encrypted (e.g. hashed) DNA sequence is not searchable. In order to check whether a particular subsequence is included, firstly the complete DNA sequence must be decompressed. Only then can a subsequence be searched for therein, which is again associated with the aforementioned high level of effort and weakens the data security.

Furthermore, it is known for a more rapid search of genetic data, to index it by means of hashing (see the publication “Bitpacking techniques for indexing genomes: I. Hash tables” by T. D. Wu in “Algorithms for Molecular Biology” (2016) 11:5). So-called “Reads” are mapped to a DNA sequence, wherein a hash table is used as a “look-up” table in which position details of corresponding subsections in the sequence are placed. In this case, the hashing enables an efficient search through a DNA sequence. However, it is present in an unencrypted form, directly readable by the user.

Further uses of hash functions are known from other fields of data processing. For example, in the encryption of passwords after a user registration during an application in a data network with a user name and a password, the password is encoded by means of a cryptographic hash function. Therein, a randomly selected character string (a “salt”) can firstly be appended to the password, so that hacking of passwords is made more difficult. The hash value determined by the encoding is stored in a database. When the user logs in to the application with his user name and his password, the password is encoded with the hash function, the hash value determined is compared with the hash value in the database and the input user name is compared with the stored user name for this password. In this use of hash functions, not only is the correct password needed for user identification, but also the correct association of user name and password. For this purpose, the user name (e.g. an email address) is available in plain text as a stored value in addition to the table entry of the hash value. In the event of hacker attacks, the user names become known directly, but the passwords are still present encoded. However, there are numerous methods for breaking passwords so that it can be assumed that if access data is obtained, for simple or often-used passwords, decoding is relatively easy. Data security is restricted by the joint storage of the user name in plain text with the hash value.

It is an objective of the invention to provide an improved method and an improved data processing apparatus for processing, in particular for encrypting and storing series of physiological and/or biological data, in particular genetic data, by means of which disadvantages of conventional techniques are avoided. The method and the data processing apparatus are intended, in particular, to enable the data to be searched more effectively and/or, in case of access restrictions, to make a search accessible without the original data being made known to third parties during a search.

This objective is achieved with a method and/or a data processing apparatus for processing genetic data, a method for querying a database, a computer program product and a computer-readable storage medium having the features of the independent claims. Advantageous embodiments and uses of the invention are disclosed by the dependent claims.

According to a first general aspect of the invention, the above objective is achieved with a method for processing genetic data which comprise a series of sequence elements, each representing a biomolecule. Preferably, the predetermined series of sequence elements comprises at least one section of genetic material, for example, exclusively coding sections, exclusively non-coding sections or both coding and non-coding sections. The biomolecules comprise, for example, nucleotides and/or amino acids. The genetic data can comprise, for example, at least one gene sequence. Alternatively, the genetic data can comprise short tandem repeat (STR) or single nucleotide polymorphism (SNP) profiles in sequence form.

Each series of sequence elements can be assigned to an individual, for example, a human or animal subject. The expression “genetic data” relates to at least one series of sequence elements. A single series of sequence elements, i.e. the genetic data of a single individual or preferably a plurality of series of sequence elements, i.e. the genetic data of a plurality of individuals can be processed. In other words, genetic data of a plurality of individuals are preferably processed, wherein the genetic data of each individual comprises a series of sequence elements, each representing a biomolecule.

Sequence fragments are formed from the genetic data of each series of sequence elements. A sequence fragment comprises a section of the series of sequence elements with a fragment length of at least two sequence elements. A coding function is applied to each of the sequence fragments in order to generate a plurality of encrypted fragment data items, each being associated with one of the sequence fragments. The coding function is a mathematical function which assigns exactly one encrypted value to each sequence fragment, represented, for example, by a succession of characters. The coding function is preferably non-invertible. The non-invertibility of the coding function means that no mathematical inverse function of the coding function exists. From the encrypted fragment data, in this embodiment of the invention, the sequence fragments are not determinable. Furthermore, the coding function is collision-resistant, i.e. two different sequence fragment inputs lead to different encrypted fragment data. Alternatively, an invertible coding function can be used, in particular for a specific use of the invention in which the data security is non-critical. The encrypted fragment data are transferred to a storage device and stored therein.

According to the invention, the formation of the sequence fragments takes place such that the sections of the series of sequence elements overlap and each sequence element is included in at least two sequence fragments. In relation to the genetic data, the sequence fragments are overlapping. Advantageously, each sequence element is thus included, together with at least one directly adjacent sequence element in the series of sequence elements in at least two sequence fragments of the sequence fragments. Each sequence fragment is encrypted. The storage in the storage device can advantageously take place without the specification of an order.

The encrypted fragment data can be stored with a random order if the order has no significance for the later querying of the storage device. The order of the encrypted fragment data is, however, retained during the storage if, in the later search in the stored data, the position of a particular search sequence within the entirety of the genetic data is also to be queried. Preferably, the encrypted fragment data are stored such that their association with the genetic data, i.e. the series of sequence elements of an individual is retained. Furthermore, the encrypted fragment data can be stored in conjunction with an item of location information. The location information contains, for example, the location of the cell material within a cell bank from which the genetic data has been obtained, or of a database in which further information regarding the cell material from which the genetic data have been obtained is stored.

With the invention, a method for encrypting genetic data is provided. The encrypted fragment data advantageously represent not only the entirety of the genetic data, but also all partial successions with the lengths of the sequence fragments formed. This enables a more effective search for successions of sequence elements in the stored encrypted fragment data. As a result, the technical effect is enabled that with a reduced expenditure of time and/or energy, it can be ascertained whether the genetic data contain a succession of sequence elements that is being searched for. It is particularly advantageous that the search can be carried out without the encryption having to be undone. The invention enables, as a further technical effect, removing of access restrictions to a database which contains the stored encrypted fragment data, without impairing data security. The information regarding the finding of searched—for data and/or the found data can be transferred unencrypted.

Although the encrypted fragment data represent the totality of the genetic data, due to the non-invertibility of the coding function, the genetic data cannot be regained from the encrypted fragment data. Due to the overlap of the sequence fragments and the optionally different fragment lengths, this will probably also not be possible in future through more efficient hacking techniques.

According to a second general aspect of the invention, the above objective is achieved by means of a data processing apparatus for processing genetic data, which is configured for generating and storing encrypted fragment data using the method according to the first general aspect of the invention or according to its different embodiments. The data processing apparatus comprises a fragmenting device which is configured to form the sequence fragments such that the sections of the series of sequence elements overlap and each sequence element is included in at least two sequence fragments, a coding device which is configured for generating the plurality of encrypted fragment data, and a storage device which is configured for storing the encrypted fragment data. The data processing apparatus is preferably realized by means of a computer. The storage device can be part of the computer or a separate database.

According to a third general aspect of the invention, the above objective is achieved by a method for querying a database which contains encrypted fragment data that has been generated and stored using the method according to the first general aspect of the invention or according to its different embodiments. The querying method comprises a specification of at least one search sequence comprising a predetermined series of sequence elements, each of which represents a biomolecule that is to be searched for, a n application of the coding function with which the encrypted fragment data have been generated on the at least one search sequence for generating at least one encrypted search sequence and a search for the at least one encrypted search sequence in the stored encrypted fragment data. If the search result is positive, a response can be returned to the user that the search sequence has been found, together with an item of information regarding in which genetic data or in which sample the search sequence has been found without the drawing of inferences regarding a particular person being possible.

The search can be directed to at least one of the following search queries, for example to determine data that is typical for a particular disease pattern:
Is the search sequence included in the encrypted fragment data?
Is the search sequence included in a particular gene section represented by the encrypted fragment data?
Is a combination and/or a logical linking (e.g. Seq 1 AND Seq 2 NOT Seq 3) of a plurality of search sequences present?
Where is biological cell material from which the genetic data have been obtained (localization function)?

The invention has the substantial advantage that the complete genetic data, such as a complete DNA sequence, does not have to be present again after the encoding in order to be able nevertheless to answer biologically or medically interesting questions. For example, it can be ascertained whether a particular disease-related mutation is included within a DNA sequence without explicitly naming this DNA sequence.

As distinct from the compression according, for example, to A. Mehta et al., according to the invention, it is not abutting, but rather overlapping sequence fragments that are generated. The inventors have found that although the scope of the data is enlarged, the search for a particular succession of sequence elements is more effective. As distinct from the indexing of genetic data according to T. D. Wu, according to the invention, exclusively encrypted data are stored.

According to a preferred embodiment of the invention, the fragment length of each sequence fragment is at least 3. Advantageously, most search queries, in particular most biologically or medically interesting questions can thus be covered after the occurrence of successions of biomolecules without the encoding and storage effort enlarging excessively.

According to a particular preferred embodiment of the invention, the formation of the sequence fragments takes place by a step-wise readout of sections of successive sequence elements from the genetic data with a progression of the readout by one step for each new section (formation of the sequence fragments with a window sliding with a step width of 1). After specification of a fragment length and of a start element in the genetic data, the sequence fragments are each provided by the sections of the series of sequence elements with the predetermined fragment length beginning at the start element and all the subsequent sequence elements. Advantageously, thereby, for each partial succession of sequence elements of the respective length, an associated sequence fragment is generated from the genetic data regardless of the position within the sequence.

With the querying of a database according to the third general aspect of the invention, when the search sequence is specified, a shortening of an initial search sequence to a search sequence length can be provided that is equal to the fragment length of the sequence fragments from which the encrypted fragment data has been generated. Thereby, the length of the search sequence is advantageously adapted to the length of the segment fragments mapped to the encrypted fragment data.

Preferably, all the sequence fragments have the same length (number of sequence elements). Thereby, a systematic, even coverage of the genetic data is ensured.

Alternatively, the sequence fragments may have different lengths. According to this alternative embodiment of the invention with different fragment lengths, the sequence fragments can form a plurality of fragment groups of sequence fragments, wherein the sequence fragments in each fragment group each have the same length, the sequence fragments of different fragment groups have different lengths, and the formation of the sequence fragments takes place such that within each fragment group, the sections of the series of sequence elements overlap and each sequence element is included in at least two sequence fragments. On application of the hash function as a coding function, each fragment group provides a hash value table. This embodiment has the particular advantage that the database with the stored encrypted fragment data can be searched through for the occurrence of search sequences with different lengths, so that the querying of the database can offer an enhanced information yield. The occurrence of a search sequence of freely selectable length (within the lengths of the sequence fragments of the fragment groups) can be found in the genetic data without knowing the genetic data. The fragment length can be greater than 3, e.g. up to 20 or more. The fragment groups from sequence fragments can be selected, for example, for a hierarchically ordered structure of the stored data. With a hierarchically ordered structure of the genetic data, for example, nested arrays of data and/or clusters can be generated which are based on fragment sizes or so-called B-trees.

According to a further, particularly advantageous embodiment of the invention, the coding function is a hash function and the encrypted fragment data are hash vales. The hash function maps sequence fragments, i.e. successions of sequence elements of a freely selectable length, specifically non-invertibly, in each case, to one hash value. The use of the hash function for encryption has particular advantages since hash functions are available and well investigated and are non-invertible so that the decryption of the genetic data from the encrypted fragment data is precluded or extremely difficult. The encoding of the genetic data of an individual provides the encrypted fragment data in the form of hash values. The hash values of an individual are stored in a database, for example, in the form of a hash value table. The database correspondingly preferably comprises a plurality of hash value tables.

In order to increase the data security, the hash function preferably has at least one of the following properties:
the hash function is a cryptographic hash function (it is advantageously collision-resistant, so that obtaining an identical hash value for two different inputs it is practically precluded),
the hash function generates hash values with a length that amounts to at least 128 bits,
the hash function meets at least the SHA2 (secure hash algorithms) standard, and
the hash function is configured for an avalanche effect such that even small changes to the input generate a completely different hash value.

According to a further embodiment of the invention, it can be advantageous if a stochastically selected character string is added to each of the sequence fragments before applying the coding function. Advantageously, by means of the addition, for example a n attachment of the randomly selected character string (“salt”), the input entropy can be increased before the further processing of the input. Alternatively or additionally, the hash function can be applied multiple times on the sequence fragments and/or the encrypted fragment data. Advantageously, the drawing of inferences from the hash value to the input by brute force methods is thereby made more difficult.

According to a further advantageous variant of the invention, the encrypted fragment data are stored in a database. The database is a storage device in which fragment data are stored, preferably encrypted according to the invention, relating to a plurality of individuals, from one or more facilities at which genetic data are obtained, e.g. clinical facilities and/or laboratories. The database is configured for access by users. Free access, for example, via a network or access with user data restricted to particular users can be enabled.

A computer program product stored on a computer-readable storage medium and configured for forming the sequence fragments and for generating a plurality of encrypted fragment data in the method according to the first general aspect of the invention, a computer-readable storage medium on which a computer program product is stored which is configured for forming the sequence fragments and for generating the plurality of encrypted fragment data in the method according to the first general aspect of the invention, and a database with a plurality of searchable encrypted fragment data that have been generated with the method according to the first general aspect of the invention are further independent subject matter of the invention.

As a further independent subject matter of the invention, a system comprising at least one facility for preparing anonymized genetic data, for example, clinical facilities and/or laboratories, and at least one facility for using the data by at least one operator, for example, a university or industrial research facility is provided.

Further details and advantages of the invention are described below, making reference to the accompanying drawings, which show in:

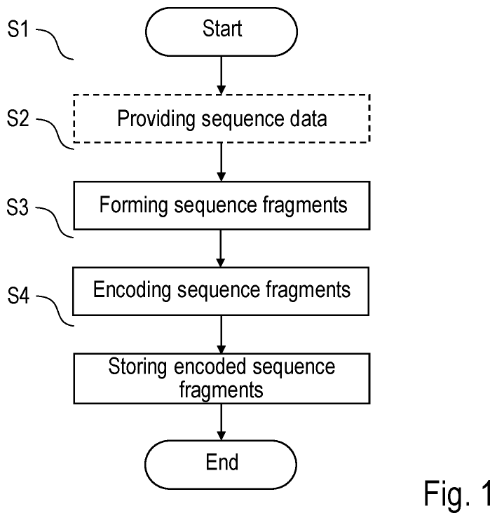

FIG. 1 a schematic illustration of the processing of genetic data according to preferred embodiments of the invention,

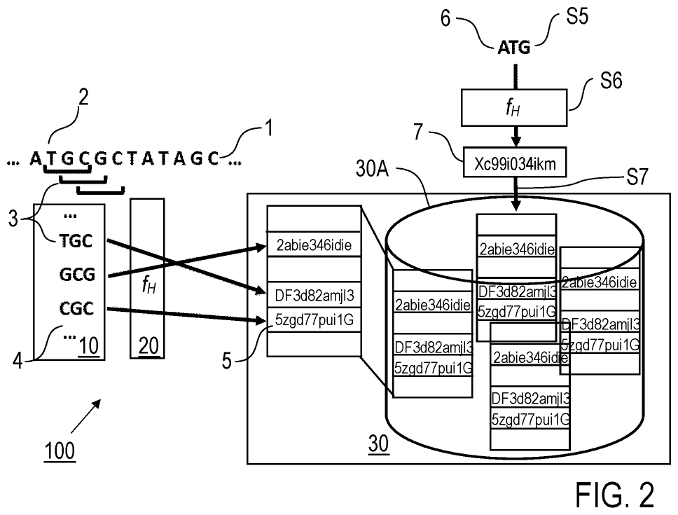

FIG. 2 further details of the encryption and storage of genetic data and the querying of a database according to further embodiments of the invention, and

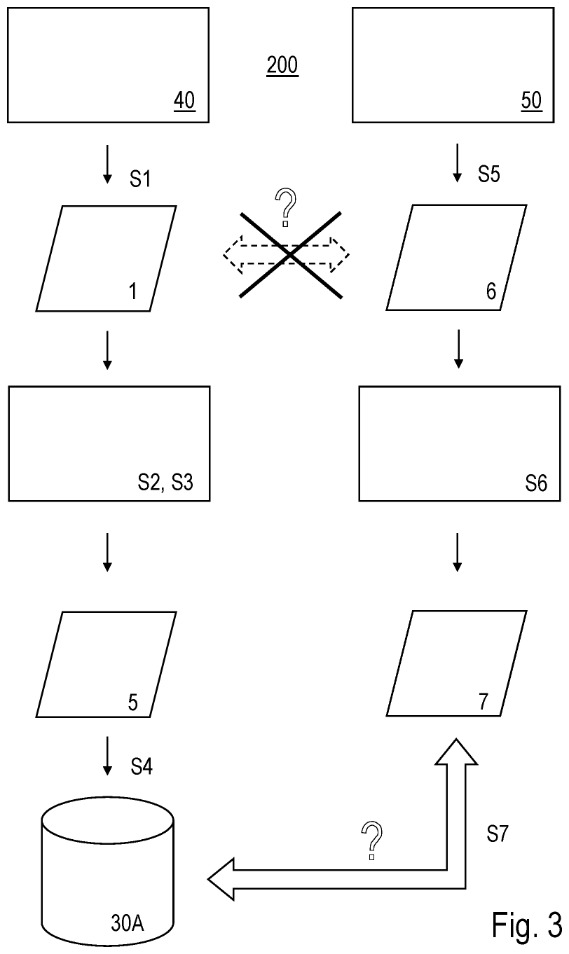

FIG. 3 a schematic overview of a preferred use of the invention for the processing of clinically obtained genetic data and their searching by users.

Details of preferred embodiments of the invention are described below, in particular in relation to the formation of the sequence fragments, their encoding and storage in a database and the querying of the database. Details of the selection of a coding function, in particular a hash function are not explained since they are known per se from conventional encoding techniques in bioinformatics or from other technical fields. Reference is made, by way of example, to the use of the invention in the processing of genetic data which comprise a nucleotide sequence. The use of the invention is not restricted to these data, but is also possible with other genetic data, such as for example amino acid sequences (protein sequences).

FIG. 1 schematically shows the main steps of the method for processing genetic data according to preferred embodiments of the invention, wherein further details are set out, by way of example, in FIG. 2. FIG. 2 also shows schematically the components of a data processing apparatus 100 with a fragmenting device 10, a coding device 20 and a storage device 30/database 30A.

In the method sequence according to FIG. 1, firstly the preparation of the genetic data 1 is shown with step S1. The preparation of the genetic data 1 comprises, for example, the sequencing of genetic material of at least one individual. The sequencing takes place using per se known sequencing techniques. Alternatively, the preparation of the genetic data 1 comprises the retrieval of genetic data 1 from existing data sources, for example, freely accessible databases. The genetic data 1 typically comprises parts of a genome of the individual, but can also represent the entire genome. For example, the genetic data 1 of a particular individual relates to genetic data of iPS cells (induced pluripotent stem cells) of the individual.

Step S1 is a preparation step of the method according to the invention. The preparation of the genetic data 1 in step S1 can be provided immediately before the subsequent processing with the steps S2 to S4 or temporally separated from them.

In step S2, the formation of the sequence fragments 3 from the genetic data 1 follows. FIG. 2 shows, by way of example, genetic data 1 from sequence elements in the form of a nucleotide sequence. The nucleotide sequence consists of the nucleobases adenine, thymine, guanine and cytosine which are usually abbreviated as A, T, G and C. As sequence fragments 3, k-mers (herein, e.g. k=3) are formed. Beginning with a start element 2 (e.g. T), the step-wise readout of sequence fragments 3 of length 3 takes place. The provision of the sequence fragments 3 takes place with a readout using a sliding window. As a result, the succession 4 of sequence fragments 3 is formed. Step S2 can be implemented with a per se known sliding window algorithm.

Subsequently, at step S3, the encoding of the sequence fragments 3 takes place with a coding device 20. The coding device 20 is configured to an application of a hash function ƒ_{H} on the sequence fragments 3. As a result of the application of the hash function, a hash value table is obtained. The elements of the hash value table are encrypted fragment data 5 which represent the sequence fragments 3. This hash value table thus contains the genome sequence of a person in a form that permits no drawing of inferences to the identity of the person, or the like.

As distinct from the representation in FIG. 2, the single application of the hash function ƒ_{H} can be replaced by the repeated (at least twofold) application of the hash function ƒ_{H} in a first application to the sequence fragments 3 and in at least one further application to the encrypted fragment data 5.

The encoding of the sequence fragments 3 provides the encrypted fragment data 5 in the hash value table. The encrypted fragment data 5 (encoded sequence fragments) are subsequently stored at step S4 in the storage device 30, for example, the database 30A. The database 30A is part of the data processing apparatus 100 or is provided separately therefrom. The encrypted fragment data 5 of a hash value table, i.e. of an individual, are stored in each case, in predetermined storage sections and/or together with a sequence identification (sample ID) representing the assignment to a particular hash value table, so that the association of the encrypted fragment data 5 with an anonymized sample from an individual is maintained.

For querying of the database 30A, as shown in the right-hand part of FIG. 2, a search sequence 6 of nucleic acids, for example ATG, is initially prepared (step S5) and, by applying the hash function, is encrypted (step S6). As a result, an encrypted search sequence 7 is prepared in the form of a hash value. Subsequently, the database is searched with regard to the occurrence of this hash value using per se known search techniques (step S7). When the encrypted search sequence 7 is located, the hash value table to which the found search sequence belongs is acquired. By means of the data structure of the database 30A with a plurality of hash value tables, this search needs a constant runtime and is therefore efficient.

Further details of a preferred use of the invention are shown in FIG. 3. With this use, a system 200 for preparing anonymized genetic data by clinical facilities and/or laboratories and for use of the data by an operator, for example, a university or industrial research facility is provided. On the left-hand side of FIG. 3 it is shown schematically how genetic data 1 are prepared, for example, at a clinical facility 40 (step S1). In a practical example, the system 200 can comprise a plurality of operators and a plurality of users who commonly access the database or a plurality of databases. Subsequently, the genetic data 1 is subjected to the method according to the invention with the steps S2 and S3 in order to prepare the encoded sequence fragments 5 and to store them in the database 30A (step S4).

A research facility 50 has an interest in an evaluation of the genetic data 1. For example, in the search for a particular disease, the question arises of whether a prepared search sequence 6 (step S5) is included in the genetic data 1 (see upper double-headed arrow). However, this direct query is made difficult or even precluded by the excessive effort for a search in the genetic data 1 and by the data protection. In order to be able nevertheless to search the genetic data 1, as described above, the search sequence 6 is subjected to the encoding for generating a hash value (step S6), after which a search can be carried out in the database 30A (step S7). If the search has the result that the stored encrypted fragment data 5 include the searched—for encrypted search sequence 7, the associated genetic data 1, i.e. the dataset of a particular individual is identified. Subsequently, a query relating to this special dataset can be placed by the research facility 50 back to the clinical facility 40 in order, while observing the rules of data security, to obtain further information regarding the individual with the relevant search sequence and/or cell material of the individual with the relevant search sequence, for example, from a cell bank.

It should be noted that the example given represents only one possible use of the invention in which it is enabled, without exact knowledge of the genetic data, to be able to process particular questions from the field of personalized medicine. Dependent upon the available data and/or the data format, only the necessary format of the search sequences and/or the search question are defined in order to prepare a hash-value match of the same data points in the database.

A further example for the use of the invention is where a research facility wishes to investigate a particular disease and for this purpose needs cell material with particular genetic features from a cell bank. If the genetic data of the material stored in the cell bank are processed according to the present invention, the invention can be applied to find suitable cell lines from the cell bank without accessing the genetic data. The research facility obtains information, with a significantly reduced cost and time expenditure, regarding which cell line is needed to carry out the planned investigations without having to sequence the cell material itself.

The features of the invention disclosed in the above description, the drawings and the claims can be significant either individually or in combination or sub-combination for the realization of the invention in its various embodiments.

1. A method for processing genetic data which comprise a series of sequence elements which represent, in each case, a biomolecule, comprising the steps
forming sequence fragments, wherein each sequence fragment comprises a section of the series of sequence elements with a fragment length of at least two sequence elements,
applying a coding function to each of the sequence fragments in order to generate a plurality of encrypted fragment data items, each being associated with one of the sequence fragments, and
storing the encrypted fragment data, wherein
the step of forming the sequence fragments takes place such that the sections of the series of sequence elements overlap and each sequence element is included in at least two sequence fragments.

2. The method according to claim 1, wherein
the fragment length of each sequence fragment at least 3.

3. The method according to claim 1, wherein the step of forming the sequence fragments comprises
specifying the fragment length and a start element in the genetic data, and
providing the sequence fragments, in each case, using the sections of the series of sequence elements with the predetermined fragment length beginning at the start element and at all the subsequent sequence elements.

4. The method according to claim 1, wherein
all the sequence fragments have the same length.

5. The method according to claim 1, wherein
the sequence fragments form a plurality of fragment groups of sequence fragments, wherein
the sequence fragments in each fragment group each have the same length,
the sequence fragments of different fragment groups have different lengths, and
the forming the sequence fragments takes place such that in each fragment group the sections of the series of sequence elements overlap and each sequence element is included in at least two sequence fragments.

6. The method according to claim 1, wherein
the coding function is a hash function and the encrypted fragment data include hash values.

7. The method according to claim 1, wherein the step of forming the sequence fragments before the application of the coding function comprises
addition, in each case, of a stochastically selected character string to each of the sequence fragments.

8. The method according to claim 1, wherein
genetic data from a plurality of individuals are processed, wherein the genetic data of each individual comprise a series of sequence elements which represent, in each case, a biomolecule.

9. A data processing apparatus which is configured for generating and storing encrypted fragment data with the method according to claim 1, comprising
a fragmenting device which is configured for forming the sequence fragments such that the sections of the series of sequence elements overlap and each sequence element is included in at least two sequence fragments,
a coding device which is configured for generating the plurality of encrypted fragment data, and
a storage device which is configured for storing the encrypted fragment data.

10. A computer program product which is stored on a computer-readable storage medium and is configured for forming the sequence fragments end for generating the plurality of encrypted fragment data in a method according to claim 1.

11. A computer-readable storage medium on which a computer program product is stored which is configured for forming the sequence fragments and for generating the plurality of encrypted fragment data in a method according to claim 1.

12. A database with a plurality of searchable, encrypted fragment data which have been generated with a method according to claim 1.

13. A method for querying a database containing encrypted fragment data which have been generated and stored with a method according to claim 1, comprising the steps
specifying a search sequence comprising a predetermined series of sequence elements which represent, in each case, a biomolecule,
applying the coding function, with which the encrypted fragment data have been generated, on the search sequence for generating an encrypted search sequence, and
searching for the encrypted search sequence in the stored encrypted fragment data.

14. The method according to claim 13, wherein
the specifying of the search sequence comprises a shortening of an initial search sequence to a search sequence length that is equal to the fragment length of the sequence fragments from which the encrypted fragment data have been generated.

15. Method according to claim 1, wherein
the encrypted fragment data are stored in a database.

16. Method according to claim 1, wherein
the predetermined series of sequence elements comprises a section of genetic material.

17. Method according to claim 1, wherein
the genetic data represent a nucleotide sequence or an amino acid sequence.
